(19) United States
(12) Patent Application Publication
(10) Pub. No.: US 2023/0295681 A1
(43) Pub. Date: Sep. 21, 2023

(54) INDUSTRIAL FERMENTATION PROCESS FOR BACILLUS USING TEMPERATURE SHIFT

(71) Applicant: BASF SE, Ludwigshafen (DE)

(72) Inventors: Andreas Daub, Ludwigshafen (DE); Aydin Golabgir Anbarani, Ludwigshafen (DE); Tobias Klein, Ludwigshafen (DE); Michael Morweiser, Ludwigshafen (DE); Georg Benjamin Wandrey, Ludwigshafen (DE)

(21) Appl. No.: 18/018,083

(22) PCT Filed: Jul. 27, 2021
(86) PCT No.: PCT/EP2021/071056

§ 371 Date: Jan. 26, 2023

(30) Foreign Application Priority Data
Jul. 28, 2020 (EP) 20188163.8

(51) Int. Cl. C12P 21/02; C12N 9/26; C12N 9/24

(52) U.S. Cl. CPC C12P 21/02; C12N 9/2411; C12N 9/2488

(57) ABSTRACT

The present invention relates to the field of industrial fermentation. In particular, it relates to a method for cultivating a Bacillus host cell comprising the steps of (a) inoculating a fermentation medium with a Bacillus host cell comprising an expression construct for a gene encoding a protein of interest, (b) cultivating for a first cultivation phase the Bacillus host cell in said fermentation medium under conditions conducive for the growth of the Bacillus host cell and the expression of the protein of interest, wherein the cultivation of the Bacillus host cell comprises the addition of at least one feed solution and wherein the cultivation during the first cultivation phase is carried out at a first temperature, and (c) cultivating for a second cultivation phase the Bacillus host cell culture obtained in step (b) under conditions conducive for the growth of the Bacillus host cell and the expression of the protein of interest, wherein the cultivation comprises the addition of at least one feed solution and wherein the cultivation during the second cultivation phase is carried out at a second temperature, said second temperature being higher than the first temperature. The invention also provides for a Bacillus host cell culture obtainable by the said method.

The present invention relates to the field of industrial fermentation. In particular, it relates to a method for cultivating a Bacillus host cell comprising the steps of (a) inoculating a fermentation medium with a Bacillus host cell comprising an expression construct for a gene encoding a protein of interest, (b) cultivating for a first cultivation phase the Bacillus host cell in said fermentation medium under conditions conducive for the growth of the Bacillus host cell and the expression of the protein of interest, wherein the cultivation of the Bacillus host cell comprises the addition of at least one feed solution and wherein the cultivation during the first cultivation phase is carried out at a first temperature, and (c) cultivating for a second cultivation phase the Bacillus host cell culture obtained in step (b) under conditions conducive for the growth of the Bacillus host cell and the expression of the protein of interest, wherein the cultivation comprises the addition of at least one feed solution and wherein the cultivation during the second cultivation phase is carried out at a second temperature, said second temperature being higher than the first temperature. The invention also provides for a Bacillus host cell culture obtainable by the said method.

Microorganisms are widely used as industrial workhorses for the production of a product of interest, especially proteins, and in particular enzymes. The biotechnological production of the product of interest is conducted via fermentation and subsequent purification of the product. Microorganisms, like the Bacillus species, are capable of secreting significant amounts of product into the fermentation broth. This allows a simple product purification process compared to intracellular production and explains the success of Bacillus in industrial application.

Industrial bioprocesses using microorganisms are typically performed in large-scale production bioreactors having a size of more than 50 m³. For the fermentation process in said large-scale bioreactors, typically, inoculation of the fermentation broth in the bioreactor is carried out with a pre-culture of Bacillus cells. A pre-culture can be obtained by cultivating Bacillus cells in smaller seed fermenters.

The large-scale fermentation process usually comprises growing the inoculated Bacillus cells under conditions which allow for growth and expression of the protein of interest to be produced. Typically, Bacillus cells are grown in complex or defined fermentation media and carbon sources will be fed in constant or varying amounts during cultivation.

Different approaches have been reported aiming at increasing the yield of protein of interest produced by the Bacillus cells during said cultivation in large scale bioreactors. These approaches concerned, e.g., variations in the composition of media. Other approaches concerned a decrease in temperature, inter alia, for reducing the likelihood of inclusion body formation (Hashemi 2012, Food Bioprocess Technol 5:1093-1099; Wenzel 2011, Applied and Environmental Microbiology 77: 6419-6425).

However, means for further increasing yield in large-scale industrial fermentation processes are highly desired.

The technical problem underlying the present invention may be seen as the provision of means and methods for complying with the aforementioned needs. It can be solved by the embodiments characterized in the claims and herein below.

Thus, the present invention relates to a method for cultivating a Bacillus host cell comprising the steps of
(a) inoculating a fermentation medium with a Bacillus host cell comprising an expression construct for a gene encoding a protein of interest;
(b) cultivating for a first cultivation phase the Bacillus host cell in said fermentation medium under conditions conducive for the growth of the Bacillus host cell and the expression of the protein of interest, wherein the cultivation of the Bacillus host cell comprises the addition of at least one feed solution and wherein the cultivation during the first cultivation phase is carried out at a first temperature; and
(c) cultivating for a second cultivation phase the Bacillus host cell culture obtained in step (b) under conditions conducive for the growth of the Bacillus host cell and the expression of the protein of interest, wherein the cultivation comprises the addition of at least one feed solution and wherein the cultivation during the second cultivation phase is carried out at a second temperature, said second temperature being higher than the first temperature.

It is to be understood that as used in the specification and in the claims, “a” or “an” can mean one or more, depending upon the context in which it is used. Thus, for example, reference to “a cell” can mean that at least one cell can be utilized.

Further, it will be understood that the term “at least one” as used herein means that one or more of the items referred to following the term may be used in accordance with the invention. For example, if the term indicates that at least one feed solution shall be used this may be understood as one feed solution or more than one feed solutions, i.e. two, three, four, five or any other number of feed solutions. Depending on the item the term refers to the skilled person understands as to what upper limit the term may refer, if any.

The term “about” as used herein means that with respect to any number recited after said term an interval accuracy exists within in which a technical effect can be achieved. Accordingly, about as referred to herein, preferably, refers to the precise numerical value or a range around said precise numerical value of ±20%, preferably ±15%, more preferably ±10%, or even more preferably ±5%.

The term “comprising” as used herein shall not be understood in a limiting sense. The term rather indicates that more than the actual items referred to may be present, e.g., if it refers to a method comprising certain steps, the presence of further steps shall not be excluded. However, the term also encompasses embodiments where only the items referred to are present, i.e. it has a limiting meaning in the sense of “consisting of”.

The present invention, thus, provides for a method that can be applied for culturing Bacillus host cells in both, laboratory and industrial scale fermentation processes. “Industrial fermentation” as referred to in accordance with the present invention refers to a cultivation method in which at least 200 g of a carbon source per liter of initial fermentation medium will be added, typically the carbon source is referred to as primary carbon source. Preferably, the primary carbon source is defined as the main source of carbon consumed by the host cell.

The “main source of carbon” or “main carbon source” typically refers to the carbon source that represents the main source of carbon based on the mass proportions of carbohydrates and/or carbon sources present during cultivation, typically present in the feed solution and/or the initial fermentation medium, more typically in the first and/or second cultivation phase and/or subsequent cultivation phases. The term “carbon source” is typically understood as the compound metabolized by an organism as the source of carbon for building its biomass and/or its growth. Suitable carbon sources include for example organic compounds such as carbohydrates.

The method according to the present invention may also comprise further steps. Such further steps may encompass the termination of cultivating and/or obtaining a product such as the protein of interest from the Bacillus host cell culture by appropriate purification techniques. Preferably, the method of the invention further comprises the step of obtaining the protein of interest from the Bacillus host cell culture obtained after step (c).

The term “cultivating” or “cultivation” as used herein refers to keeping alive and/or propagating Bacillus cells comprised in a culture at least for a predetermined time. The term encompasses phases of exponential cell growth at the beginning of growth after inoculation as well as phases of stationary growth.

In the method of the present invention, a fermentation medium is inoculated with a Bacillus host cell comprising an expression construct for a gene encoding a protein of interest as a first step.

The term “inoculating” as used herein refers to introducing Bacillus host cells into the fermentation medium used cultivation. Inoculation of the fermentation medium with the Bacillus host cells can be achieved by introducing Bacillus host cells of a pre-culture (starter culture). Preferably, the fermentation is inoculated with pre-culture that has been grown under conditions known to the person skilled in the art. The pre-culture can be obtained by cultivating the cells in a pre-culture medium that can be a chemically defined pre-culture medium or a complex pre-culture medium. The pre-culture medium can be the same or different from the fermentation medium used for cultivation in the method of the present invention. The complex pre-culture medium can contain complex nitrogen and / or complex carbon sources. Preferably, the pre-culture used for inoculation is obtained by using a complex culture medium. The pre-culture can be added all or in part to the main fermentation medium. Preferably, the Bacillus host cells in the pre-culture are actively growing cells, i.e. they are in a stage where the number of cells is increasing. Typically, cells in a pre-culture are upon inoculation of the pre-culture in a lag phase and switch over time to a phase of exponential growth. Preferably, cells in the exponential growth phase are used for from the pre-culture for inoculation of the fermentation medium. The volume ratio between pre-culture used for inoculation and main fermentation medium is, preferably, between 0.1 and 30 % (v/v).

The term “Bacillus host cell” refers to a Bacillus cell which serves as a host for an expression construct for a gene encoding a protein of interest. Said expression construct may be a naturally occurring expression construct, a recombinantly introduced expression construct or a naturally occurring expression construct which has been genetically modified in the Bacillicus cell. The Bacillius host cell may be a host cell from any member of the bacterial genus Bacillus, preferably a host cell of Bacillus licheniformis, Bacillus subtilis, Bacillus alkalophilus, Bacillus amyloliquefaciens, Bacillus brevis, Bacillus circulans, Bacillus clausii, Bacillus coagulans, Bacillus firmus, Bacillus jautus, Bacillus lentus, Bacillus megaterium, Bacillus pumilus, Bacillus stearothermophilus, Bacillus thuringiensis or Bacillus velezensis. More preferably, the Bacillus host cell is a Bacillus licheniformis, Bacillus pumilus, or Bacillus subtilis host cell, even more preferred Bacillus licheniformis or Bacillus subtilis host cell, most preferably, Bacillus licheniformis host cell. Particular preferably, the Bacillus licheniformis is selected from the group consisting of Bacillus licheniformis as deposited under American Type Culture Collection number ATCC 14580, ATCC 31972, ATCC 53757, ATCC 53926, ATCC 55768, and under DSMZ number (German Collection of Microorganisms and Cell Cultures GmbH) DSM 13, DSM 394, DSM 641, DSM 1913, DSM 11259, and DSM 26543.

Typically, the host cell belongs to the species Bacillus licheniformis, such as a host cell of the Bacillus licheniformis strain ATCC 14580 (which is the same as DSM 13, see Veith et al. “The complete genome sequence of Bacillus licheniformis DSM 13, an organism with great industrial potential.” J. Mol. Microbiol. Biotechnol. (2004) 7:204-211). Alternatively, the host cell may be a host cell of Bacillus licheniformis strain ATCC 53926. Alternatively, the host cell may be a host cell of Bacillus licheniformis strain ATCC 31972. Alternatively, the host cell may be a host cell of Bacillus licheniformis strain ATCC 53757. Alternatively, the host cell may be a host cell of Bacillus licheniformis strain ATCC 53926. Alternatively, the host cell may be a host cell of Bacillus licheniformis strain ATCC 55768. Alternatively, the host cell may be a host cell of Bacillus licheniformis strain DSM 394. Alternatively, the host cell may be a host cell of Bacillus licheniformis strain DSM 641. Alternatively, the host cell may be a host cell of Bacillus licheniformis strain DSM 1913. Alternatively, the host cell may be a host cell of Bacillus licheniformis strain DSM 11259. Alternatively, the host cell may be a host cell of Bacillus licheniformis strain DSM 26543.

The Bacillus host cell to be applied in the method of the present invention shall comprise an expression construct for a gene encoding a protein of interest to be expressed by the said host cell. The term “expression construct” as referred to herein refers to a polynucleotide comprising a nucleic acid sequence encoding the protein of interest operably linked to an expression control sequence, e.g., a promoter. Typically, the expression construct as used in the method according to the invention may at least comprise a nucleic acid sequence encoding the protein of interest operably linked to a promoter.

A promoter as referred to herein is a nucleotide sequence located upstream of a gene on the same strand as the gene that enables transcription of said gene. The activity of a promoter (also referred to as promoter activity) is understood herein as the capacity of the promoter to enable and initiate transcription of said gene, in other words it is understood as the capacity of the promoter to drive gene expression. The promoter is followed by the transcription start site of the gene. The promoter is recognized by an RNA polymerase, typically, together with the required transcription factors, which initiate transcription. A functional fragment or functional variant of a promoter is a nucleotide sequence which is recognizable by RNA polymerase and is capable of initiating transcription. Functional fragments or functional variants of promoters are also encompassed as a promoter in the sense of the present invention.

Promoters may be inducer-dependent promoters the activity of which depend on an activating signal molecule, i.e., the presence of an inducer molecule, or may be inducer-independent promoters, i.e. promoters that do not depend on the presence of an inducer molecule added to or present in the fermentation medium and that are either constitutively active or can be increased in activity regardless of the presence of an inducer molecule that is present in or added to the fermentation medium. Preferably, the promoter is an inducer-independent promoter. Typically, the host cell has not been genetically modified in its ability to take up or metabolize an inducer molecule, preferably, wherein the host cell is not manP and/ or manA deficient.

Preferably, the promoter is selected from the group consisting of the promoter sequences of the aprE promoter (a native promoter from the gene encoding the Bacillus subtilisin Carlsberg protease), amyQ promoter from Bacillus amyloliquefaciens, amyL promoter and variants thereof from Bacillus licheniformis (preferably as de-scribed in US5698415), bacteriophage SPO1 promoter, such as the promoter PE4, PE5, or P15 (preferably as described in WO2015118126 or in Stewart, C. R., Gaslightwala, I., Hinata, K., Krolikowski, K. A., Needleman, D. S., Peng, A. S., Peterman, M. A., Tobias, A., and Wei, P. 1998, Genes and regulatory sites of the “host-takeover module” in the terminal redundancy of Bacillus subtilis bacteriophage SPO1. Virology 246(2), 329-340), crylllA promoter from Bacillus thuringiensis (preferably as described in WO9425612 or in Agaisse, H. and Lereclus, D. 1994. Structural and functional analysis of the promoter region involved in full expression of the crylllA toxin gene of Bacillus thuringiensis. Mol.Microbiol. 13(1). 97-107.), and combinations thereof, and active fragments or variants thereof.

Preferably, the promoter sequences can be combined with 5′-UTR sequences native or heterologous to the host cell, as described herein. Preferably, the promoter is an inducer-independent promoter. More preferably, the promoter is selected from the group consisting of: an veg promoter, lepA promoter, serA promoter, ymdA promoter, fba promoter, aprE promoter, amyQ promoter, amyL promoter, bacteriophage SPO1 promoter, cryIIIA promoter, combinations thereof, and active fragments or variants thereof. Even more preferably, the promoter sequence is selected from the group consisting of aprE promoter, amyL promoter, veg promoter, bacteriophage SPO1 promoter, and cryIIIA promoter, and combinations thereof, or active fragments or variants thereof. Still even more preferably, the promoter is selected from the group consisting of: an aprE promoter, SPO1 promoter, such as PE4, PE5, or P15 (preferably as described in WO15118126), tandem promoter comprising the promoter sequences amyl and amyQ (preferably as described in WO9943835), and triple promoter comprising the promoter sequences amyL, amyQ, and cryllla (preferably as described in WO2005098016). Most preferably, the promoter is an aprE promoter, preferably, an aprE promoter from Bacillus amyloliquefaciens, Bacillus clausii, Bacillus haloduans, Bacillus lentus, Bacillus licheniformis, Bacillus pumilus, Bacillus subtilis, or Bacillus velezensis, more preferably from Bacillus licheniformis, Bacillus pumilus or Bacillus subtilis, most preferably, from Bacillus licheniformis.

Utilizing an inducer-independent promoter as specified herein above may be advantageous as it allows for continuous expression of the gene of interest throughout the fermentation resulting in a continuous and stable protein production without the need of an inducer molecule. Hence, utilizing an inducer-independent promoter may contribute to improve the yield of the protein of interest.

It will be understood that the activity of the promoter used in accordance with the method of the present invention, preferably, is not dependent on heat-inducible elements. Accordingly, the promoter to be used as an expression control sequence in accordance of the present invention, preferably, is a temperature-insensitive promoter and/or lacks a heat-inducible element.

In contrast, thereto an “inducer-dependent promoter” is understood herein as a promoter that is increased in its activity to enable transcription of the gene to which the promoter is operably linked upon addition of an “inducer molecule” to the fermentation medium. Thus, for an inducer-dependent promoter the presence of the inducer molecule triggers via signal transduction an increase in expression of the gene operably linked to the promoter. The gene expression prior activation by the presence of the inducer molecule does not need to be absent, but can also be present at a low level of basal gene expression that is increased after addition of the inducer molecule. The “inducer molecule” is a molecule which presence in the fermentation medium is capable of affecting an increase in expression of a gene by increasing the activity of an inducer-dependent promoter operably linked to the gene. Inducer molecules known in the art include carbohydrates or analogs thereof, that may function as secondary carbon source in addition to a primary carbon source such as glucose. Typically, the Bacillus host cell has not been genetically modified in its ability to take up or metabolize an inducer molecule, more typically the Bacillus host cell is not manP and/or manA deficient.

Preferably, the method for cultivating according to the present invention occurs without the addition of a secondary carbon source such as mannose, sucrose, β-glucosides, oligo-β-glucosides, fructose, mannitol, lactose, allolactose, isopropyl-β-D-1-thiogalactopyranoside (IPTG), L-arabinose, xylose. Even more preferred, the fermentation medium is free of any secondary carbon source.

Moreover, said expression construct may comprise further elements required for proper termination of translation or elements required for insertion, stabilization, introduction into a host cell or replication of the said expression construct. Such sequences encompass, inter alia, 5′-UTR (also called leader sequence), ribosomal binding site (RBS, Shine-Dalgarno sequence), 3′-UTR, transcription start and stop sites and, depending on the nature of the expression construct, origin of replications, integration sites, and the like. Preferably, the nucleic acid construct and / or the expression vector comprises a 5′-UTR and a RBS. Preferably, the 5′-UTR is selected from the control sequence of a gene selected from the group consisting of aprE, grpE, ctoG, SP82, gsiB, crylla and ribG gene.

Yet, the expression construct shall also comprise a nucleic acid sequence encoding a protein of interest. The “protein of interest” as referred to herein refers to any protein, peptide or fragment thereof which is intend to be produced in the Bacillus host cell. A protein, thus, encompasses polypeptides, peptides, fragments thereof as well as fusion proteins and the like.

Preferably, the protein of interest is an enzyme. In a particular embodiment, the enzyme is classified as an oxidoreductase (EC 1), a transferase (EC 2), a hydrolase (EC 3), a lyase (EC 4), an isomerase (EC 5), or a ligase (EC 6) (EC-numbering according to Enzyme Nomenclature, Recommendations (1992) of the Nomenclature Committee of the International Union of Biochemistry and Molecular Biology including its supplements published 1993-1999). In a preferred embodiment, the protein of interest is an enzyme suitable to be used in detergents.

More preferably, the enzyme is a hydrolase (EC 3), even more preferably, or a glycosidase (EC 3.2), still even more preferably a glycosidase (EC 3.2). Especially preferred enzymes are enzymes selected from the group consisting of an amylase (in particular an alpha-amylase (EC 3.2.1.1)), a cellulase (EC 3.2.1.4), a lactase (EC 3.2.1.108), a mannanase (EC 3.2.1.25), a lipase (EC 3.1.1.3), a phytase (EC 3.1.3.8), and a nuclease (EC 3.1.11 to EC 3.1.31); in particular an enzyme selected from the group consisting of amylase, lipase, mannanase, phytase, xylanase, phosphatase, glucoamylase, nuclease, and cellulase, preferably, amylase or mannanase. Still even more preferably the enzyme is a glycosidase (EC 3.2) selected from mannanases and amylases.

Preferably, the protein of interest is secreted into the fermentation medium. Secretion of the protein of interest into the fermentation medium typically allows for a facilitated separation of the protein of interest from the fermentation medium. For secretion of the protein of interest into the fermentation medium the nucleic acid construct may comprise a polynucleotide encoding for a signal peptide that directs secretion of the protein of interest into the fermentation medium. Various signal peptides are known in the art. Preferred signal peptides are selected from the group consisting of the signal peptide of the AprE protein from Bacillus subtilis or the signal peptide from the YvcE protein from Bacillus subitilis.

Particularly suitable for secreting enzymes, such as amylases, from Bacillus cells into the fermentation medium are the signal peptide of the AprE protein from Bacillus subtilis or the signal peptide from the YvcE protein from Bacillus subtilis. As the YvcE signal peptide is suitable for secreting a wide variety of different enzymes, including amylases, this signal peptide can be used, preferably in conjunction with the fermentation process described herein.

It will be understood that each of the expression control sequence, nucleic acid sequence encoding the protein of interest and/or the aforementioned further elements may be from the Bacillus host cell or may be from another species, i.e. heterologous with respect to said Bacillus host cell.

Further, the expression construct may be an arrangement of a gene of interest and the expression control sequence and/or further elements as specified before which is native to, i.e., endogenously present in the genome of the Bacillus host cell. Moreover, the term also encompasses such native expression constructs which have been genetically manipulated, e.g., by genomic editing and/or mutagenesis technologies.

The expression construct may also be an exogenously introduced expression construct. In an exogenously introduced expression construct, the expression control sequence, the gene encoding the protein of interest and/or the further elements may be native with respect to the host cell or may be derived from other species, i.e. be heterologous with respect to the Bacillus host cell. The introduction of the expression construct into a Bacillus host cell can be accomplished in accordance with the present invention by any method known in the art, including, inter alia, well known transformation, transfection, transduction, and conjugation techniques and the like. Preferably, the expression construct exogenously introduced is comprised in a vector, preferably, an expression vector. The expression vector can be, preferably, located outside the chromosomal DNA of the Bacillus host cell, i.e. be present episomally, in one or more copies. However, the expression vector may also preferably be integrated into the chromosomal DNA of the Bacillus cell in one or more copies. The expression vector can be linear or circular. Preferably, the expression vector is a viral vector or a plasmid.

For autonomous replication, the expression vector may further comprise an origin of replication enabling the vector to replicate autonomously in the host cell in question. Bacterial origins of replication include but are not limited to the origins of replication of plasmids pUB110, pC194, pTB19, pAMβ1, and pTA1060 permitting replication in Bacillus (Janniere, L., Bruand, C., and Ehrlich, S.D. (1990). Structurally stable Bacillus subtilis cloning vectors. Gene 87, 53-6; Ehrlich, S.D., Bruand, C., Sozhamannan, S., Dabert, P., Gros, M.F., Janniere, L., and Gruss, A. (1991). Plasmid replication and structural stability in Bacillus subtilis. Res. Microbiol. 142, 869-873), and pE194 (Dempsey, L.A. and Dubnau, D.A. (1989). Localization of the replication origin of plasmid pE194. J. Bacteriol. 171, 2866-2869). The origin of replication may be one having a mutation to make its function temperature-sensitive in the host cell (see, e.g., Ehrlich, 1978, Proceedings of the National Academy of Sciences USA 75:1433-1436). Yet, the expression vector, preferably, contains one or more selectable markers that permit easy selection of transformed Bacillus host cells. A selectable marker is a gene encoding a product, which provides for biocide resistance, resistance to heavy metals, prototrophy to auxotrophs, and the like. Bacterial selectable markers include but are not limited to the dal genes from Bacillus subtilis or Bacillus licheniformis, or markers that confer antibiotic resistance such as ampicillin, kanamycin, erythromycin, chloramphenicol or tetracycline resistance. Furthermore, selection may be accomplished by co-transformation, e.g., as described in WO9109129, where the selectable marker is on a separate vector.

The method of the present invention further comprises the step of cultivating for a first cultivation phase the Bacillus host cell in said fermentation medium under conditions conducive for the growth of the Bacillus host cell and the expression of the protein of interest, wherein the cultivation of the Bacillus host cell comprises the addition of at least one feed solution and wherein the cultivation during the first cultivation phase is carried out at a first temperature.

The term “first cultivation phase” as used herein refers to a first period of time for which cultivation at a first temperature is to be carried out. Said period of time may be pre-determined or variable dependent on parameters of the culture, e.g., bacterial growth rates, carbon source consumption rates, amount of carbon source which has been provided to the fermentation medium or the like. Said at least one feed solution shall provide a carbon source at increasing rates, preferably exponentially increasing rates. Preferably, said at least one feed solution provides a primary carbon source comprising a carbohydrate during the fermentation, typically in a first cultivation phase and/or in a second cultivation phase. More preferably, the primary carbon source is glucose. Said period of time may be pre-determined or variable dependent on parameters of the culture, e.g., bacterial growth rates, carbon source consumption rates, amount of carbon source which has been provided to the fermentation medium or the like. Preferably, said first cultivation phase is carried out for a time of at least about 3 h up to about 48 h, preferably for about 22 h. Alternatively, it may be carried out until a pre-determined total amount of carbon source has been provided by the at least one feed solution. Preferably, the at least one feed solution provides a carbon source at exponentially increasing rates with an exponential factor of at least about 0.13 h⁻¹ and a starting amount of at least about 1 g per liter and hour of the at least one carbon source. Further preferably, a total amount of at least about 50 g or more of said at least one carbon source per kg Bacillus host cell culture being initially present in step b) is added during the first cultivation phase. Further details are to be found in the accompanying Examples, below. The skilled person is well aware of how to determine the time period of the first cultivation period. The Bacillus host cell is cultivated in said first cultivation phase under conditions which allow for the growth of the Bacillus host cell and the expression of the protein of interest.

The term “fermentation medium” as used herein refers to a water-based solution containing one or more chemical compounds that can support the growth of cells. Preferably, the fermentation medium according to the present invention is a complex fermentation medium or a chemically defined fermentation medium.

A complex fermentation medium as used to herein refers to a fermentation medium that comprise a complex nutrient source in an amount of 0.5 to 30% (w/v) of the fermentation medium. Complex nutrient sources are nutrient sources which are composed of chemically undefined compounds, i.e., compounds that are not known by their chemical formula, preferably comprising undefined organic nitrogen- and/or carbon-containing compounds. In contrast thereto, a “chemically defined nutrient source” (e.g., “chemically defined carbon source” or “chemically defined nitrogen source”) is understood to be used for nutrient sources which are composed of chemically defined compounds. A chemically defined component is a component which is known by its chemical formula. A complex nitrogen source is a nutrient source that is composed of one or more chemically undefined nitrogen containing compounds, i.e., nitrogen containing compounds that are not known by their chemical formula, preferably comprising organic nitrogen containing compounds, e.g., proteins and/or amino acids with unknown composition. A complex carbon source is a carbon source that is composed of one or more chemically undefined carbon containing compounds, i.e., carbon containing compounds that are not known by their chemical formula, preferably comprising organic carbon containing compounds, e.g., carbohydrates with unknown composition. It is clear for the skilled person that a complex nutrient source might be a mixture of different complex nutrient sources. Thus, a complex nitrogen source can comprise a complex carbon source and vice versa and a complex nitrogen source can be metabolized by the cells in a way that it functions as carbon source and vice versa.

Preferably, the complex nutrient source is a complex nitrogen source. Complex sources of nitrogen include, but are not limited to protein-containing substances, such as an extract from microbial, animal or plant cells, e.g., plant protein preparations, soy meal, corn meal, pea meal, corn gluten, cotton meal, peanut meal, potato meal, meat, casein, gelatins, whey, fish meal, yeast protein, yeast extract, tryptone, peptone, bacto-tryptone, bacto-peptone, wastes from the processing of microbial cells, plants, meat or animal bodies, and combinations thereof. In one embodiment, the complex nitrogen source is selected from the group consisting of plant protein, preferably potato protein, soy protein, corn protein, peanut, cotton protein, and/or pea protein, casein, tryptone, peptone and yeast extract and combinations thereof.

Preferably, the fermentation medium may also comprise defined media components. Preferably, the fermentation medium also comprises a defined nitrogen source. Examples of inorganic nitrogen sources are ammonium, nitrate, and nitrite, and combinations thereof. In a preferred embodiment, the fermentation medium comprises a nitrogen source, wherein the nitrogen source is a complex or a defined nitrogen source or a combination thereof. In one embodiment, the defined nitrogen source is selected from the group consisting of ammonia, ammonium, ammonium salts, (e.g., ammonium chloride, ammonium nitrate, ammonium phosphate, ammonium sulfate, ammonium acetate), urea, nitrate, nitrate salts, nitrite, and amino acids, preferably, glutamate, and combinations thereof.

Preferably, the complex nutrient source is in an amount of 2 to 15% (v/w) of the fermentation medium. In another embodiment, the complex nutrient source is in an amount of 3 to 10% (v/w) of the fermentation medium.

Also preferably, the complex fermentation medium may further comprise a carbon source. The carbon source is, preferably, a complex or a defined carbon source or a combination thereof. Preferably, the complex nutrient source comprises a carbohydrate source. Various sugars and sugar-containing substances are suitable sources of carbon, and the sugars may be present in different stages of polymerization. Preferred complex carbon sources to be used in the present invention are selected from the group consisting of molasse, corn steep liquor, cane sugar, dextrin, starch, starch hydrolysate, and cellulose hydrolysate, and combinations thereof. Preferred defined carbon sources are selected from the group consisting of carbohydrates, organic acids, and alcohols, preferably, glucose, fructose, galactose, xylose, arabinose, sucrose, maltose, lactose, acetic acid, propionic acid, lactic acid, formic acid, malic acid, citric acid, fumaric acid, glycerol, inositol, mannitol and sorbitol, and combinations thereof. Preferably, the defined carbon source is provided in form of a syrup, which can comprise up to 20%, preferably, up to 10%, more preferably up to 5% impurities. In one embodiment, the carbon source is sugar beet syrup, sugar cane syrup, corn syrup, preferably, high fructose corn syrup. In another embodiment, the complex carbon source is selected from the group consisting of molasses, corn steep liquor, dextrin, and starch, or combinations thereof, and wherein the defined carbon source is selected from the group consisting of glucose, fructose, galactose, xylose, arabinose, sucrose, maltose, dextrin, lactose, or combinations thereof.

Preferably, the fermentation medium is a complex medium comprising complex nitrogen and complex carbon sources. More preferably, the fermentation medium is a complex medium comprising complex nitrogen and carbon sources, wherein the complex nitrogen source may be partially hydrolyzed as described in WO 2004/003216.

Yet, the fermentation medium may, typically, also comprises a hydrogen source, an oxygen source, a sulfur source, a phosphorus source, a magnesium source, a sodium source, a potassium source, a trace element source, and a vitamin source as further described elsewhere herein.

In another embodiment, the fermentation medium may be a chemically defined fermentation medium. A chemically defined fermentation medium is a fermentation medium which is essentially composed of chemically defined components in known concentrations. A chemically defined component is a component which is known by its chemical formula. A fermentation medium which is essentially composed of chemically defined component includes a medium which does not contain a complex nutrient source, in particular, no complex carbon and/or complex nitrogen source, i.e., which does not contain complex raw materials having a chemically undefined composition. A fermentation medium which is essentially composed of chemically defined components may further include a medium which comprises an essentially small amount of a complex nutrient source, for instance a complex nitrogen and/or carbon source, an amount as defined below, which typically is not sufficient to maintain growth of the Bacillus host cells and/or to guarantee formation of a sufficient amount of biomass.

In that regard, complex raw materials have a chemically undefined composition due to the fact that, for instance, these raw materials contain many different compounds, among which complex heteropolymeric compounds, and have a variable composition due to seasonal variation and differences in geographical origin. Typical examples of complex raw materials functioning as a complex carbon and/or nitrogen source in fermentation are soybean meal, cotton seed meal, corn steep liquor, yeast extract, casein hydrolysate, molasses, and the like. An essentially small amount of a complex carbon and/or nitrogen source may be present in the chemically defined fermentation medium according to the invention, for instance as carry-over from the inoculum for the main fermentation. The inoculum for the main fermentation is not necessarily obtained by fermentation on a chemically defined medium. Most often, carry-over from the inoculum will be detectable through the presence of a small amount of a complex nitrogen source in the chemically defined fermentation medium of the main fermentation. Small amounts of a complex medium components, like complex carbon and/or nitrogen source, might also be introduced into the fermentation medium by the addition of small amounts of these complex components to the fermentation medium. It may be advantageous to use a complex carbon and/or nitrogen source in the fermentation process of the inoculum for the main fermentation, for instance to speed up the formation of biomass. i.e. to increase the growth rate of the microorganism, and/or to facilitate internal pH control. For the same reason, it may be advantageous to add an essentially small amount of a complex carbon and/or nitrogen source, e.g. yeast extract, to the initial stage of the main fermentation, especially to speed up biomass formation in the early stage of the fermentation process. An essentially small amount of a complex nutrient source which may be added to the chemically defined fermentation medium in the fermentation process according to the invention is defined to be an amount of at the most 10% of the total amount of the respective nutrient, which is added in the fermentation process. In particular, an essentially small amount of a complex carbon and/or nitrogen source which may be added to the chemically defined fermentation medium is defined to be an amount of a complex carbon source resulting in at the most 10% of the total amount of carbon and/or an amount of a complex nitrogen source resulting in at the most 10% of the total amount of nitrogen, which is added in the fermentation process, preferably an amount of a complex carbon source resulting in at the most 5% of the total amount of carbon and/or an amount of a complex nitrogen source resulting in at the most 5% of the total amount of nitrogen, more preferably an amount of a complex carbon source resulting in at the most 1 % of the total amount of carbon and/or an amount of a complex nitrogen source resulting in at the most 1 % of the total amount of nitrogen, which is added in the fermentation process. Preferably, at the most 10% of the total amount of carbon and/or at the most 10% of the total amount of nitrogen, preferably an amount of at the most 5% of the total amount of carbon and/or an amount of at the most 5% of the total amount of nitrogen, more preferably an amount of at the most 1 % of the total amount of carbon and/or an amount of at the most 1 % of the total amount of nitrogen which is added in the fermentation process is added via carry-over from the inoculum. Most preferably, no complex carbon and/or complex nitrogen source is added to the fermentation medium in the fermentation process.

A chemically defined nutrient source as referred to herein e.g., chemically defined carbon source or chemically defined nitrogen source, is understood to be used for nutrient sources which are composed of chemically defined compounds.

Culturing a microorganism in a chemically defined fermentation medium requires that cells be cultured in a medium which contain various chemically defined nutrient sources selected from the group consisting of chemically defined hydrogen source, chemically defined oxygen source, chemically defined carbon source, chemically defined nitrogen source, chemically defined sulfur source, chemically defined phosphorus source, chemically defined magnesium source, chemically defined sodium source, chemically defined potassium source, chemically defined trace element source, and chemically defined vitamin source. Preferably, the chemically defined carbon source is selected from the group consisting of carbohydrates, organic acids, hydrocarbons, alcohols and mixtures thereof. Preferred carbohydrates are selected from the group consisting of glucose, fructose, galactose, xylose, arabinose, sucrose, maltose, maltotriose, lactose, dextrin, maltodextrins, starch and inulin, and mixtures thereof. Preferred alcohols are selected from the group consisting of glycerol, methanol and ethanol, inositol, mannitol and sorbitol and mixtures thereof. Preferred organic acids are selected from the group consisting of acetic acid, propionic acid, lactic acid, formic acid, malic acid, citric acid, fumaric acid and higher alkanoic acids and mixtures thereof. Preferably, the chemically defined carbon source comprises glucose or sucrose. More preferably, the chemically defined carbon source comprises glucose, even more preferably the predominant amount of the chemically defined carbon source is provided as glucose.

Most preferably, the chemically defined carbon source is glucose. As indicated elsewhere herein, glucose may be the preferred primary carbon source. It is to be understood that the chemically defined carbon source can be provided in form of a syrup, preferably as glucose syrup. As understood herein, glucose as referred to herein shall include glucose syrups. A glucose syrup is a viscous sugar solution with high sugar concentration. The sugars in glucose syrup are mainly glucose and to a minor extent also maltose and maltotriose in varying concentrations depending on the quality grade of the syrup. Preferably, besides glucose, maltose and maltotriose the syrup can comprise up to 10%, preferably, up to 5%, more preferably up to 3% impurities. Preferably, the glucose syrup is from corn.

The chemically defined nitrogen source is preferably selected from the group consisting of urea, ammonia, nitrate, nitrate salts, nitrite, ammonium salts such as ammonium chloride, ammonium sulphate, ammonium acetate, ammonium phosphate and ammonium nitrate, and amino acids such as glutamate or lysine and combinations thereof. More preferably, a chemically defined nitrogen source is selected from the group consisting of ammonia, ammonium sulphate and ammonium phosphate. Most preferably, the chemically defined nitrogen source is ammonia. The use of ammonia as a chemically defined nitrogen source has the advantage that ammonia additionally can function as a pH controlling agent.

Additional compounds can be added in complex and chemically defined fermentation medium as described below.

Oxygen is usually provided during the cultivation of the cells by aeration of the fermentation media by stirring and/or gassing. Hydrogen is usually provided due to the presence of water in the aqueous fermentation medium. However, hydrogen and oxygen are also contained within the carbon and/or nitrogen source and can be provided that way.

Magnesium can be provided to the fermentation medium by one or more magnesium salts, preferably selected from the group consisting of magnesium chloride, magnesium sulfate, magnesium nitrate, magnesium phosphate, and combinations thereof, or by magnesium hydroxide, or by combinations of one or more magnesium salts and magnesium hydroxide.

Sodium can be added to the fermentation medium by one or more sodium salts, preferably selected from the group consisting of sodium chloride, sodium nitrate, sodium sulphate, sodium phosphate, sodium hydroxide, and combinations thereof.

Calcium can be added to the fermentation medium by one or more calcium salts, preferably selected from the group consisting of calcium sulphate, calcium chloride, calcium nitrate, calcium phosphate, calcium hydroxide, and combinations thereof.

Potassium can be added to the fermentation medium in chemically defined form by one or more potassium salts, preferably selected from the group consisting of potassium chloride, potassium nitrate, potassium sulphate, potassium phosphate, potassium hydroxide, and combinations thereof.

Phosphorus can be added to the fermentation medium by one or more salts comprising phosphorus, preferably selected from the group consisting of potassium phosphate, sodium phosphate, magnesium phosphate, phosphoric acid, and combinations thereof. Preferably, at least 1 g of phosphorus is added per liter of initial fermentation medium.

Sulfur can be added to the fermentation medium by one or more salts comprising sulfur, preferably selected from the group consisting of potassium sulfate, sodium sulfate, magnesium sulfate, sulfuric acid, and combinations thereof.

Preferably, the fermentation medium and/or the initial fermentation medium, comprises one or more selected from the group consisting of:
0.1 to 50 g nitrogen per liter of fermentation medium;
1 to 6 g phosphorus per liter of fermentation medium;
0.15 to 2 g sulfur per liter of fermentation medium;
0.4 to 8 g potassium per liter of fermentation medium;
0.01 to 2 g sodium per liter of fermentation medium;
0.01 to 3 g calcium per liter of fermentation medium; and
0.1 to 10 g magnesium per liter of fermentation medium.

Typically, the feed solution differs from the fermentation medium and/or from the initial fermentation medium, in one or more of the compounds of said group listed above. Even more typically, the feed solution differs from the fermentation medium and/or from the initial fermentation medium, in the amount of one or more of the compounds of said group listed above.

One or more trace element ions can be added to the fermentation medium, preferably in amounts of below 10 mmol/L initial fermentation medium each. These trace element ions are selected from the group consisting of iron, copper, manganese, zinc, cobalt, nickel, molybdenum, selenium, and boron and combinations thereof. Preferably, the trace element ions iron, copper, manganese, zinc, cobalt, nickel, and molybdenum are added to the fermentation medium. Preferably, the one or more trace element ions are added to the fermentation medium in an amount selected from the group consisting of 50 µmol to 5 mmol per liter of initial medium of iron, 40 µmol to 4 mmol per liter of initial medium copper, 30 µmol to 3 mmol per liter of initial medium manganese, 20 µmol to 2 mmol per liter of initial medium zinc, 1 µmol to 100 µmol per liter of initial medium cobalt, 2 µmol to 200 µmol per liter of initial medium nickel, and 0.3 µmol to 30 µmol per liter of initial medium molybdenum, and combinations thereof. For adding each trace element preferably one or more from the group consisting of chloride, phosphate, sulphate, nitrate, citrate and acetate salts can be used.

Compounds which may optionally be included in the fermentation medium are chelating agents, such as citric acid, MGDA, NTA, or GLDA, and buffering agents such as mono- and dipotassium phosphate, calcium carbonate, and the like. Buffering agents preferably are added when dealing with processes without an external pH control. In addition, an antifoaming agent may be dosed prior to and/or during the fermentation process.

Vitamins refer to a group of structurally unrelated organic compounds, which are necessary for the normal metabolism of cells. Cells are known to vary widely in their ability to synthesize the vitamins they require. A vitamin should be added to the fermentation medium of Bacillus cells not capable of synthesizing said vitamin. Vitamins can be selected from the group of thiamin, riboflavin, pyridoxal, nicotinic acid or nicotinamide, pantothenic acid, cyanocobalamin, folic acid, biotin, lipoic acid, purines, pyrimidines, inositol, choline and hemins.

Preferably, the fermentation medium also comprises a selection agent, e.g., an antibiotic, such as ampicillin, tetracycline, kanamycin, hygromycin, bleomycin, chloroamphenicol, streptomycin or phleomycin, to which the selectable marker of the cells provides resistance.

The amount of necessary compounds to be added to the medium will mainly depend on the amount of biomass which is to be formed in the fermentation process. The amount of biomass formed may vary widely, typically the amount of biomass is from about 10 to about 150 grams of dry cell mass per liter of fermentation broth. Usually, for protein production, fermentations producing an amount of biomass which is lower than about 10 g of dry cell mass per liter of fermentation broth are not considered industrially relevant.

The optimum amount of each component of a defined medium, as well as which compounds are essential and which are non-essential, will depend on the type of Bacillus cell which is subjected to fermentation in a medium, on the amount of biomass and on the product to be formed. Typically, the amount of medium components necessary for growth of the microbial cell may be determined in relation to the amount of carbon source used in the fermentation, typically in relation to the main carbon source, since the amount of biomass formed will be primarily determined by the amount of carbon source used.

Particular preferred fermentation media are also described in the Examples below.

Preferably, the fermentation medium is sterilized prior to use in order to prevent or reduce growth of microorganisms during the fermentation process, which are different from the inoculated microbial cells. Sterilization can be performed with methods known in the art, for example but not limited to, autoclaving or sterile filtration. Some or all medium components can be sterilized separately from other medium components to avoid interactions of medium components during sterilization treatment or to avoid decomposition of medium components under sterilization conditions.

The phrase “conditions conducive for the growth of the Bacillus host cell and the expression of the protein of interest” means that conditions other than the temperature or fermentation medium used for cultivation. Such conditions comprise pH during cultivation, physical movement of the culture by shaking or stirring and/or atmospheric conditions applied to the culture.

The pH of the fermentation medium during cultivation may be adjusted or maintained. Preferably, the pH of the medium is adjusted prior to inoculation. Preferred pH values envisaged for the fermentation medium are within the range of about pH 6.6 to about pH 9, preferably within the range of about pH 6.6 to about pH 8.5, more preferably within the range of about pH 6.8 to about pH 8.5, most preferably within the range of about pH 6.8 to about pH 8.0. As an example, for a Bacillus cell host cell culture, the pH is, preferably, adjusted to or above about pH 6.8, about pH 7.0, about pH 7.2, about pH 7.4, or about pH 7.6. Preferably, the pH of the fermentation medium during cultivation of the Bacillus host cell culture is adjusted to a PH within the rage of about pH 6.8 to about pH 9, preferably about pH 6.8 to about pH 8.5, more preferably about pH 7.0 to about pH 8.5, most preferably about pH 7.2 to about pH 8.0.

Physical movement can be applied by stirring and/or shaking of the fermentation medium. Preferably, said stirring of the fermentation medium is carried out with about 50 to about 2000 rpm, preferably with about 50 to about 1600 rpm, further preferred with about 800 to about 1400 rpm, more preferably with about 50 to about 200 rpm.

Besides stirring, oxygen and/or other gases may be applied to the culture by adjusting suitable atmospheric conditions. Preferably, oxygen is supplied with 0 to 3 bar air or oxygen.

Furthermore, additional conditions including the selection of suitable bioreactors or vessels for cultivation of Bacillus host cells are well known in the art and can be made by the skilled artisan without further ado.

The term “feed solution” as used herein refers to a solution that is added to the fermentation medium after inoculation of the initial fermentation medium with Bacillus host cells. The initial fermentation medium typically refers to the fermentation medium present in the fermenter at the time of inoculation with the Bacillus host cells. The feed solution comprises compounds supportive for the growth of said cells. Compared to the fermentation medium the feed solution may be enriched for one or more compounds.

A feed medium or feed solution used e.g. when the culture is run in fed-batch mode may be any of the above mentioned medium components or combination thereof. It is understood herein that at least part of the compounds that are provided as feed solution can already be present to a certain extent in the fermentation medium prior to feeding of said compounds.. Preferably, said feed solution provides a primary carbon source comprising at least one carbohydrate, typically in a first cultivation phase and/or in a second cultivation phase. More preferably, the carbohydrate comprised in the feed solution represents the main source of carbon consumed or metabolized by the host cell. Still more preferably, the feed solution comprises a chemically defined carbon source, preferably, glucose. Even more preferably, the feed solution comprises 40% to 60% glucose, preferably 42% to 58% glucose, more preferably 45% to 55% glucose, even more preferably 47% to 52% glucose and most preferably 50% glucose. Even more preferably, glucose is the main carbon source present in the feed solution and/or in the fermentation medium. Typically, the same feed solution may be used for the seed fermenter run in fedbatch mode and the production bioreactor. The feed solution used for the seed fermenter run in fedbatch mode may differ from the feed solution used in the production bioreactor. However, the feed solution used for the seed fermenter run in fedbatch mode and the feed solution used in the production bioreactor may have the same concentration of glucose, but the feed solution used in the production bioreactor contains salts which are not present in the feed solution used for the seed fermenter run in fedbatch mode.

Various feed profiles are known in the art. A feed solution can be added continuously or discontinuously during the fermentation process. Discontinuous addition of a feed solution can occur once during the fermentation process as a single bolus or several times with different or same volumes. Continuous addition of a feed solution can occur during the fermentation process at the same or at varying rates (i.e., volume per time). Also combinations of continuous and discontinuous feeding profiles can be applied during the fermentation process. Components of the fermentation medium that are provided as feed solution can be added in one feed solution or as different feed solutions. In case more than one feed solution is applied, the feed solutions can have the same or different feed profiles as described above.

Particular preferred feed solutions are also described in the Examples below.

The term “first temperature” as referred to herein means a temperature which is used for cultivating the Bacillus host cell culture during the first cultivation phase. It will be understood that the first temperature is constantly applied during the first cultivation phase. Moreover, the first temperature shall be a temperature which allows for the growth of the Bacillus host cell and the expression of the protein of interest. Preferably, said first temperature is within the range of about 28° C. to about 32° C., about 29° to about 31° C., preferably, is about 30° C.

The method of the present invention further comprises the step of cultivating for a second cultivation phase the Bacillus host cell culture obtained in the previous step under conditions conducive for the growth of the Bacillus host cell and the expression of the protein of interest, wherein the cultivation comprises the addition of at least one feed solution and wherein the cultivation during the second cultivation phase is carried out at a second temperature, said second temperature being higher than the first temperature.

The term “second cultivation phase” as used herein refers to a second period of time for which cultivation at a second temperature is to be carried out. Said period of time may be predetermined or variable dependent on parameters of the culture, e.g., bacterial growth rates, carbon source consumption rates, amount of carbon source which has been provided to the fermentation medium or the like. Said at least one feed solution shall provide a carbon source at a constant rate, at decreasing rates or at rates increasing less than the rates applied during the first cultivation phase. However, said constant rate or the starting rate of said decreasing rates or the staring rate of said rates increasing less than the rates in step (b) is below the maximum rate of the first cultivation phase. Preferably, the degree of increase in the rates of carbon source provided by a feed solution as referred to herein can be determined by comparing individual or constantly applied feed solution amounts and determining, e.g., a factor for the said increase. By comparing the increase factors in the first and second cultivation phase for the carbon source provided by the feed solution, it can be determined whether said carbon source is provided in the second cultivation phase at rates increasing less than in the first cultivation phase. Said second period of time may be pre-determined or variable dependent on parameters of the culture, e.g., bacterial growth rates, carbon source consumption rates, amount of carbon source which has been provided to the fermentation medium or the like. In the second cultivation phase there shall be constant growth of the Bacillus host cell culture when the at least one feed solution provides a carbon source at a constant rate. Preferably, said second cultivation phase is carried out for a time of at least about 3 h up to about 120 h, of at least about 3 h up to about 96 h, of at least about 40 h up to about 120 h or, preferably, at least about 40 h up to about 96 h. The skilled person is well aware of how to determine the time period of the second cultivation period. Preferably, the at least one feed solution provides the carbon source at a constant rate which is, preferably, within the range of about 70% to about 20%, preferably, within the range of about 50% to about 30% or, more preferably, about 35% of the maximum feeding rate for the at least one carbon source applied in the first cultivation phase. The Bacillus host cell is cultivated in said second cultivation phase under conditions which allow for the growth of the Bacillus host cell and the expression of the protein of interest.

The term “second temperature” as referred to herein means a temperature which is used for cultivating the Bacillus host cell culture during the second cultivation phase. It will be understood that the second temperature is constantly applied during the second cultivation phase. Moreover, the second temperature shall be a temperature which allows for the growth of the Bacillus host cell and the expression of the protein of interest. Preferably, said second temperature is within the range of about 33° C. to about 37° C., about 34° to about 36° C. or, preferably, is about 35° C.

Said second temperature shall be higher than the first temperature. Preferably, said first and said second temperature differ by about 3° C. to about 7° C., about 4° C. to about 6° C., or preferably, by about 5° C.

Preferably, the increase in temperature in the second cultivation phase viz-a-viz the first cultivation phase results in an increase in yield of the protein of interest. More preferably, the yield of the protein of interest obtained after step c) is significantly increased compared to a control which has been obtained by carrying out the method according to the invention wherein the said first and second temperature are identical. More preferably, said yield is increased by at least 40%, at least 60%, at least 80%, at least 100%, at least 200%, at least 300% or at least 400%.

The increase in yield may be determined dependent on the protein of interest by any technique which allows for specific quantification of the protein of interest. Some techniques are referred to elsewhere herein. As referred to herein, said increase is an increase compared to a control. The control is, preferably, a Bacillus host cell culture which has been cultivated by a method having the steps of the method of the invention and wherein said first and said second temperature are identical, i.e. a method without a temperature increase between step b) and step c). Accordingly, for determining an increase in yield, the amount of protein of interest is determined in Bacillus host cell culture which has been cultivated according to the method of the present invention and a control Bacillus host cell culture. Both determined amounts are compared to each other in order to calculate the increase in yield. Whether such increase in yield is statistically significant, or not, can be determined by various statistical tests well known to those skilled in the art. Typical tests are the Student’s t-test or Mann-Whitney U test.

After completion of the second cultivation phase, i.e. after step c), the Bacillus host cell culture may be further treated. Preferably, the protein of interest is obtained from said Bacillus host cell culture. More preferably, the protein of interest is obtained from the Bacillus host cell culture by purification.

Dependent on the nature of the protein of interest, a suitable technique may be selected. For example, if the protein of interest is secreted into the fermentation broth, the Bacillus cells may be separated from the culture and the protein of interest may be purified from the liquid part of the fermentation broth. If the protein of interest is a cellular protein, i.e. is present within the Bacillus host cell, it may be purified by separating the Bacillus host cells from the fermentation broth, subsequent lysis of said host cells and purification of the protein of interest from the lysed Bacillus host cells of the culture. Alternatively, the Bacillus host cells present in the culture after step c) may be lysed and the protein of interest may be purified from the lysed Bacillus host cells in the fermentation broth.

Purification of the protein of interest may dependent on the selected technique comprise steps of physical separation, such as centrifugation, evaporation, freeze-drying, filtration (in particular, ultrafiltration) electrophoresis (preparative SDS PAGE or isoelectric focusing electrophoresis) ultrasound, and/or pressure, or chemical treatments, such as chemical precipitation, crystallization, extraction and/or enzymatic treatments. Chromatography (e.g., ion exchange, hydrophobic, chromatofocusing, and size exclusion chromatography)may be applied as well. Affinity chromatography may also be used including antibody-based affinity chromatography or techniques using purification tags. Suitable techniques are well known in the art and can be applied depending on the protein of interest by the skilled artisan without further ado.

Moreover, the method of the present invention may also comprise further treatments including treatments of the protein of interest which has been purified as described before. Such treatments may comprise chemical and/or physical treatments which improve the purification such as addition of antifoaming agents or stabilizing agents for the protein of interest. The method of the invention may also encompass manufacturing steps for obtaining a commercial product or article comprising the protein of interest, in particular, capsules, granulates, powders, liquids and the like.

Preferably, the method of the present invention can be used for the manufacture of a purified or partially purified composition comprising the protein of interest. More preferably, the method of the present invention provides the protein of interest in purified or partially purified form.

Advantageously, it has been found in the experiments underlying the present invention that when cultivating Bacillus host cells for the manufacture of a protein of interest, a two phase cultivation using an increased cultivation temperature during the second phase increases the production of the protein of interest in said cultured Bacillus cells. In particular, it was found that a temperature shift of about 5° C. between the said first and said second cultivation phase was able to increase the yield in protein of interest made by the Bacillus host cells significantly and, typically and dependent on the Bacillus cell and the protein of interest, in the range of at least 40% up to at least 400% compared to control cultures which have not been subjected to the temperature shift. This effect achieved by the temperature shift shall be a general effect on gene expression in the cultured Bacillus host cells and shall be independent on the use of particular expression control sequences. Accordingly, thanks to the present invention, the yield in fermentation processes aiming at the microbiologic production of a protein of interest can be increased by a generally applicable cultivation method. Said method can be easily included into existing production schemes and merely requires the variation of a single parameter, i.e. the temperature applied during cultivation.

The explanations and interpretations of the terms made above apply mutatis mutandis to the embodiments described herein below.

The following embodiments are preferred embodiments of the method of the invention.

In a preferred embodiment of the method of the invention, said method further comprises obtaining the protein of interest from the Bacillus host cell culture obtained after step (c).

In a further preferred embodiment of the method of the invention, said first cultivation phase is carried out for a time of at least about 3 h up to about 48 h.

In a preferred embodiment of the method of the invention, during the first cultivation phase at least one feed solution provides a carbon source at increasing rates, preferably, exponentially increasing rates. Preferably, during the first cultivation phase the at least one feed solution provides a carbon source at exponentially increasing rates with an exponential factor of at least about 0.13 h⁻¹ and a starting amount of at least about 1 g of the at least one carbon source. In a preferred embodiment of the method of the present invention, said first cultivation a total amount of at least about 50 g of said at least one carbon source per kg Bacillus host cell culture being initially present in step b) is added.

In a further preferred embodiment of the method of the invention, said second cultivation phase is carried out for a time of at least about 3 h up to about 120 h, of at least about 3 h up to about 96 h, of at least about 40 h up to about 120 h or, preferably, at least about 40 h up to about 96 h.

In yet a preferred embodiment of the method of the invention, during the second cultivation phase the at least one feed solution provides a carbon source at a constant rate, at decreasing rates or at rates increasing less than the rates in step (b), wherein said constant rate or the starting rate of said decreasing rates or the staring rate of said rates increasing less than the rates in step (b) is below the maximum rate of the first cultivation phase.

In a preferred embodiment of the method of the present invention, said at least one feed solution in step (c) provides the said carbon source at a constant rate. Preferably, said constant rate is below the maximum rate of the feeding rates of the first cultivation phase. More preferably, said constant rate is within the range of about 70% to about 20%, preferably, within the range of about 50% to about 30% or, more preferably, about 35% of the maximum feeding rate for the at least one carbon source applied in the first cultivation phase.

In a preferred embodiment of the method of the invention, said first and said second temperature differ by about 3° C. to about 7° C., about 4° C. to about 6° C. or preferably, by about 5° C.

In a preferred embodiment of the method of the invention, said first temperature is within the range of about 28° C. to about 32° C., about 29° to about 31° C. or, preferably, is about 30° C.

In a further preferred embodiment of the method of the invention, said second temperature is within the range of about 33° C. to about 37° C., about 34° to about 36° C. or, preferably, is about 35° C.

In yet a further preferred embodiment of the method of the invention, the yield of the protein of interest obtained after step c) is significantly increased compared to a control which has been obtained by carrying out the method according to the invention wherein the said first and second temperature are identical. More preferably, said yield is increased by at least 40%, at least 60%, at least 80%, at least 100%, at least 200%, at least 300% or at least 400%.

In a preferred embodiment of the method of the invention, said Bacillus is selected from the group consisting of: Bacillus licheniformis, Bacillus subtilis, Bacillus alkalophilus, Bacillus amyloliquefaciens, Bacillus brevis, Bacillus circulans, Bacillus clausii, Bacillus coagulans, Bacillus firmus, Bacillus jautus, Bacillus lentus, Bacillus megaterium, Bacillus pumilus, Bacillus stearothermophilus, Bacillus thuringiensis, and Bacillus velezensis. More preferably, said Bacillus is Bacillus licheniformis, Bacillus pumilus, or Bacillus subtilis, even more preferred Bacillus is Bacillus licheniformis or Bacillus subtilis, and, even more preferably, Bacillus licheniformis.

In a still even more preferred embodiment, the host cell belongs to the species Bacillus licheniformis, such as a host cell of the Bacillus licheniformis strain ATCC 14580 (which is the same as DSM 13, see Veith et al. “The complete genome sequence of Bacillus licheniformis DSM 13, an organism with great industrial potential.” J. Mol. Microbiol. Biotechnol. (2004) 7:204-211). Alternatively, the host cell may be a host cell of Bacillus licheniformis strain ATCC 53926. Alternatively, the host cell may be a host cell of Bacillus licheniformis strain ATCC 31972. Alternatively, the host cell may be a host cell of Bacillus licheniformis strain ATCC 53757. Alternatively, the host cell may be a host cell of Bacillus licheniformis strain ATCC 53926. Alternatively, the host cell may be a host cell of Bacillus licheniformis strain ATCC 55768. Alternatively, the host cell may be a host cell of Bacillus licheniformis strain DSM 394. Alternatively, the host cell may be a host cell of Bacillus li-cheniformis strain DSM 641. Alternatively, the host cell may be a host cell of Bacillus licheniformis strain DSM 1913. Alternatively, the host cell may be a host cell of Bacillus licheniformis strain DSM 11259. Alternatively, the host cell may be a host cell of Bacillus licheniformis strain DSM 26543.

In a further preferred embodiment of the method of the invention, said expression construct for a gene encoding a protein of interest has been introduced into the Bacillus host cell by genetic modification. Preferably, said expression construct comprises one or more heterologous nucleic acids. More preferably, said expression construct is comprised in a vector, preferably, an expression vector.

In another preferred embodiment of the method of the invention, said expression construct comprises nucleic acid sequences endogenously present in said Bacillus host cell. Preferably, the expression construct is comprised in the genome of the Bacillus host cell. More preferably, said expression construct present in the genome has been genetically modified.

In another preferred embodiment of the method of the invention, said expression construct comprises an expression control sequence, e.g. a promoter, which governs expression of the gene encoding the protein of interest in said Bacillus host cell.

In another preferred embodiment of the method of the invention, the expression construct comprises at least a nucleic acid sequence encoding the protein of interest operably linked to an expression control sequence, e.g. a promoter. Preferably, said expression control sequence is a temperature-insensitive promoter. Preferably, said promoter is an inducer-independent promoter, or preferably, a constitutively active promoter. More preferably, said promoter is selected from the group consisting of: veg promoter, lepA promoter, serA promoter, ymdA promoter, fba promoter, aprE promoter, amyQ promoter, amyL promoter, bacteriophage SPO1 promoter and cryIIIA promoter or a combination of such promoters and/or active fragments or variants thereof.

In a preferred embodiment, the inducer-independent promoter is an aprE promoter.

In a preferred embodiment of the method of the present invention, said fermentation medium is a chemically defined fermentation medium.

In a preferred embodiment of the method of the invention, said fermentation medium comprises macroelements and trace elements in pre-defined amounts.

In a further preferred embodiment of the method of the invention, said at least one feed solution provides at least one chemically defined carbon source, preferably comprising a carbohydrate; more preferably the carbohydrate is glucose.

In a further preferred embodiment of the method of the present invention, the protein of interest is secreted into the fermentation medium.

In a further preferred embodiment of the method of the present invention, said protein of interest is an enzyme. Preferably, said enzyme is a hydrolase (EC 3), preferably, or a glycosidase (EC 3.2). More preferably, the enzyme is selected from the group consisting of: an amylase, in particular an alpha-amylase (EC 3.2.1.1), a cellulase (EC 3.2.1.4), a lactase (EC 3.2.1.108), a mannanase (EC 3.2.1.25), a lipase (EC 3.1.1.3), a phytase (EC 3.1.3.8), and a nuclease (EC 3.1.11 to EC 3.1.31). Still even more preferably the enzyme is a glycosidase (EC 3.2) selected from mannanases and amylases.

The present invention also provides a method for the manufacture of a protein of interest comprising the step of cultivating a Bacillus host cell according to the aforementioned method of the present invention and the further step of obtaining the protein of interest from the cultured Bacillus host cell.

The present invention also relates to a Bacillus host cell culture obtainable by the method of any one of the present invention. It will be understood that the Bacillus host cell culture comprises the protein of interest produced by the method of the present invention, preferably, in an increased amount.

The present invention also relates to a composition comprising the protein of interest obtainable by the method of the present invention.

All references cited throughout this specification are herewith incorporated by reference with respect to the specifically mentioned disclosure content and in their entireties.

### FIGURES

FIG. 1: Relative yields of amylases from fed-batch fermentations of Bacillus licheniformis at constant temperatures of 30° C. and 35° C. versus shifting temperature during fermentation from 30° C. to 35° C. Shown are two exemplified fed-batch fermentations Amylase 1 (A) and Amylase 2 (B).

FIG. 2: Relative enzyme yields from fed-batch fermentations at constant temperature and using temperature shift. (A) Relative yields of amylase 1 from fed-batch fermentations of Bacillus subtilis at constant temperatures of 30° C. versus shifting temperature during fermentation from 30° C. to 35° C. (B) Relative yields of mannanase from fed-batch fermentations of Bacillus licheniformis at constant temperatures of 30° C. versus shifting temperature during fermentation from 30° C. to 35° C.

FIG. 3: Optimizing time point of temperature shift from 30° C. to 35° C. by combining temperature shift with the reduction in the specific substrate uptake rate qs. (A) shows the glucose feed rate over the feed time. The total feed time was 70 h (corresponding to 100 %). (B) depicts the glucose feed rate over the relative amount of glucose added. (C) depicts the specific glucose uptake rate (qs) over the relative amount of glucose added. (D) depicts the amylase yield depending on the amount of total glucose added before the temperature shift. The arrow indicates the bar representing the combination of temperature shift and shift in feed rate.

### EXAMPLES

The invention will now be illustrated by working Examples. Theses working Examples must not construed, whatsoever, as limitations of the scope of the invention.

### Example 1: Shifting Temperature During Fermentation Increases Amylase Production in Bacillus Licheniformis

Unless otherwise stated the following experiments have been performed by applying standard equipment, methods, chemicals, and biochemicals as used in genetic engineering and fermentative production of chemical compounds by cultivation of microorganisms. See also Sambrook et al. (Molecular Cloning: A Laboratory Manual. 2nd edition, Cold Spring Harbor Laboratory, Cold 20 Spring Harbor Laboratory Press, Cold Spring Harbor, NY, 1989) and Chmiel et al. (Bioprocesstechnik 1. Einführung in die Bioverfahrenstechnik, Gustav Fischer Verlag, Stuttgart, 1991).

Alpha-amylase activity was determined by a method employing the substrate Ethyliden-4-nitrophenyl-α-D-maltoheptaoside (EPS). D-maltoheptaoside is a blocked oligosaccharide which can be cleaved by an endo-amylase. Following the cleavage an alpha-glucosidase liberates a PNP molecule which has a yellow color and thus can be measured by visible spectophotometry at 405 nm. Kits containing EPS substrate and alpha-glucosidase are available from Roche Costum Biotech (cat. No. 10880078t3) and are described in Lorentz K. et al. (2000), Clin. Chem., 46/5: 644 - 649. The slope of the time dependent absorption-curve is directly proportional to the specific activity (activity per mg enzyme) of the alpha-amylase in question under the given set of conditions.

Bacillus licheniformis strains expressing amylase 1 or amylase 2 were cultivated in a fermentation process using a chemically defined fermentation medium providing the components listed in Table 1 and Table 2.

TABLE 1

Macroelements provided in the fermentation process

| Compound | Formula | Added per initial mass [g/kg] |
|---|---|---|
| Citric acid Monohydrate | C₆H₈O₇ * H₂O | 11.2 |
| Calcium | Ca | 0.3 |
| Sodium | Na | 1.6 |
| Potassium | P | 4.0 |
| Magnesium | Mg | 0.4 |
| Sulfate | SO₄ | 2.9 |
| Ammonium | NH₄ | 0.3 |
| Phosphate | PO₄ | 15.8 |

TABLE 2

Trace elements provided in the fermentation process

| Compound | Symbol | Added per initial mass [µmol/kg] |
|---|---|---|
| Manganese | Mn | 240 |
| Zinc | Zn | 175 |
| Copper | Cu | 320 |
| Cobalt | Co | 11 |
| Nickel | Ni | 3 |
| Molybdenum | Mo | 20 |
| Iron | Fe | 385 |

The fermentation was started with a medium containing 8 g/l glucose. A solution containing 50% glucose was used as feed solution. The pH was adjusted during fermentation using ammonia.

The feed was started upon depletion of the initial amount of 8 g/l glucose indicated by an increase of culture pH and glucose was added until > 200 g of glucose per kg initial fermentation volume were added to the bioreactor. The glucose feeding strategy consisted of an initial exponential feed phase with an exponential factor of 0.13 h⁻¹ and a starting value of 1 g of glucose per L initial volume and hour where 28% of the total glucose were added to the bioreactor. This was followed by a second phase of constant glucose feeding with a rate corresponding to 35% of the maximum glucose feeding rate. In this second phase the rest of the glucose (72% of the total glucose) was added. pH was kept over 7.0 by addition of NH₄OH.

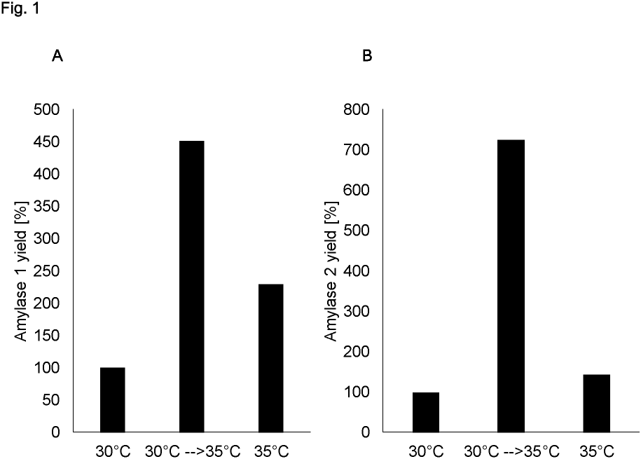

The cultivation temperature was kept constant at either 30° C. or 35° C., resulting in relative amylase yields of 100% and 229% for amylase 1 and 100% and 143% for amylase 2, respectively. Starting the fermentation at a lower temperature of 30° C. and then increasing the temperature to 35° C. after the end of the exponential feeding phase increased the yield to 451% and 723% for amylase 1 and amylase 2, respectively. Thus, performing a shift in temperature during the fermentation from a lower temperature to a higher temperature increased productivity significantly compared to fermentations where temperature was kept constant at either the lower (30° C.) or higher (35° C.) temperature. Results are depicted in FIG. 1.

### Example 2: Shifting Temperature During Fermentation Increases Amylase Production in Bacillus Subtilis

Enzyme activity was determined as described in Example 1. A Bacillus subtilis strain expressing amylase 1 was grown in mineral salt media in a fed-batch fermentation with glucose as carbon source as described in Example 1.

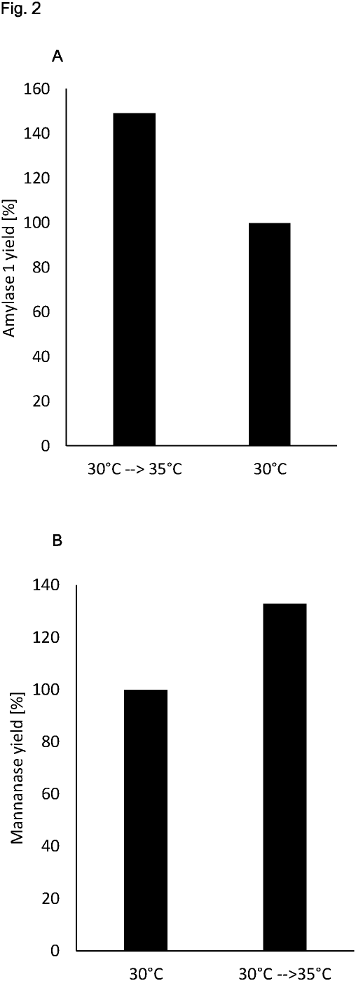

The cultivation temperature was kept constant at either 30° C. or the fermentation was started at 30° C. and then the temperature increased to 35° C. after the end of the exponential feeding phase. Performing a shift in temperature during the fermentation from a lower to a higher setpoint increased productivity significantly (49% increase) compared to fermentations where temperature was kept constant at 30° C. Results are shown in FIG. 2 (A).

### Example 3: Shifting Temperature During Fermentation Increases Mannanase Production in BacilLus Licheniformis

A mannanase molecule as described in WO2021/058453 (Seq ID No:1) was expressed in Bacillus licheniformis. The Bacillus licheniformis strain was then grown in mineral salt media in a fedbatch fermentation with glucose as carbon source as described in Example 1. The cultivation temperature was kept constant at either 30° C. or the fermentation was started at 30° C. and then the temperature increased to 35° C. after the end of the exponential feeding phase. Mannanase titers were determined from cultivation samples over the course of the fermentations by CE-SDS electrophoresis according to standard test procedures known to a person skilled in the art. Performing a shift in temperature during the fermentation from a lower to a higher setpoint increased productivity significantly (33% increase) compared to fermentations where temperature was kept constant at 30° C. Results are shown in FIG. 2 (B).

### Example 4: Combining Temperature Shift With Reduction of Specific Substrate Uptake Rate Qₛ increases amylase yield

Enzyme activity was determined as described in Example 1. A Bacillus licheniformis strain expressing amylase 1 was grown in mineral salt media in a fed-batch fermentation with glucose as carbon source as described in Example 1.

After start of the glucose feeding, the shift in temperature from 30° C. to 35° C. was performed after different amounts glucose were added (0% = start of feeding). After addition of 28% of the total amount of glucose, the feed profile was shifted from an exponential profile to a constant feed, resulting in a reduction of the specific substrate uptake rate qₛ [gram glucose per gram cells and hour] to 35% of the maximum observed during the cultivation.

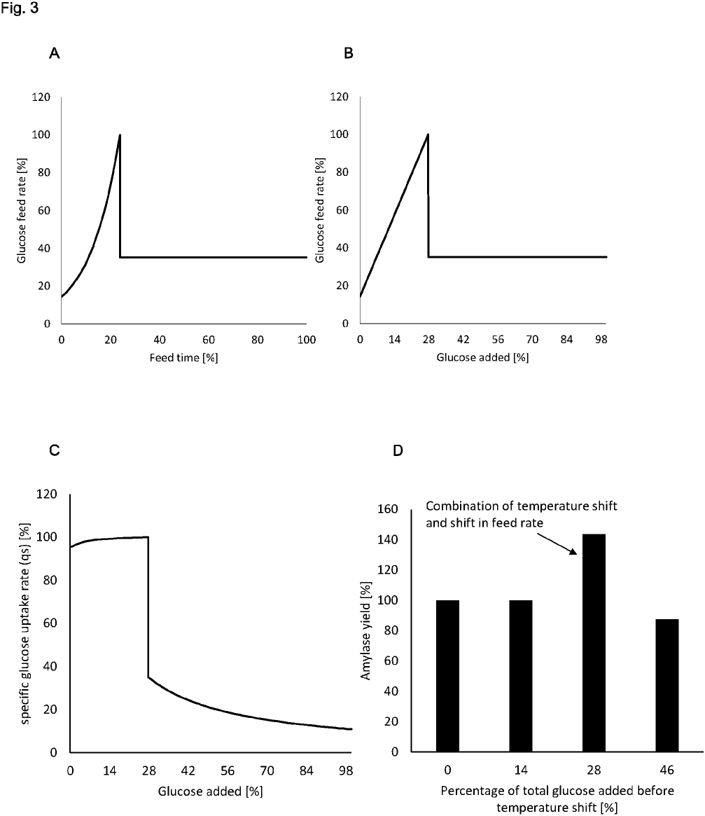

The maximum amylase yield was achieved by shifting the temperature in parallel with the switch to the constant feed rate (28% of glucose added of total amount of glucose added during the fermentation process) i.e. the reduction in the specific substrate uptake rate to 35% of its maximum. Performing the temperature shift before or after the reduction of qₛ resulted in lower product titers. Consequently, a synergetic effect was achieved by shifting cultivation temperature and qₛ at the same time. Results are shown in FIG. 3.

1. A method for cultivating a Bacillus host cell comprising the steps of
(a) inoculating a fermentation medium with a Bacillus host cell comprising an expression construct for a gene encoding a protein of interest;
(b) cultivating for a first cultivation phase the Bacillus host cell in said fermentation medium under conditions conducive for the growth of the Bacillus host cell and the expression of the protein of interest, wherein the cultivation of the Bacillus host cell comprises the addition of at least one feed solution and wherein the cultivation during the first cultivation phase is carried out at a first temperature; and
(c) cultivating for a second cultivation phase the Bacillus host cell culture obtained in step (b) under conditions conducive for the growth of the Bacillus host cell and the expression of the protein of interest, wherein the cultivation comprises the addition of at least one feed solution and wherein the cultivation during the second cultivation phase is carried out at a second temperature, said second temperature being higher than the first temperature.

2. The method of claim 1, wherein said method further comprises obtaining the protein of interest from the Bacillus host cell culture obtained after step (c).

3. The method of claim 1, wherein the protein of interest is an enzyme.

4. The method of claim 1, wherein the expression construct comprises a nucleic acid sequence encoding the protein of interest operably linked to a promoter.

5. The method of claim 1, wherein said first cultivation phase is carried out for a time of at least about 3 h up to about 48 h.

6. The method of claim 1, wherein during the first cultivation phase the at least one feed solution provides a carbon source at increasing rates.

7. The method of claim 1, wherein said second cultivation phase is carried out for a time of at least about 3 h up to about 96 h.

8. The method of claim 1, wherein during the second cultivation phase the at least one feed solution provides a carbon source at a constant rate, at decreasing rates or at rates increasing less than the rates in step (b), wherein said constant rate or the starting rate of said decreasing rates or the staring rate of said rates increasing less than the rates in step (b) is below the maximum rate of the first cultivation phase.

9. The method of claim 1, wherein said first and said second temperature differ by about 3° C. to about 7°C.

10. The method of claim 1, wherein said first temperature is within the range of about 28° C. to about 32 °C.

11. The method of claim 1, wherein said second temperature is within the range of about 33° C. to about 37 °C.

12. The method of claim 1, wherein the yield of the protein of interest obtained after step c) is significantly increased compared to a control which has been obtained by carrying out the method wherein the said first and second temperature are identical.

13. The method of claim 12, wherein said yield is increased by at least 40%, at least 60%, at least 80%, at least 100%, at least 200%, at least 300% or at least 400%.

14. The method of claim 1, wherein said Bacillus is selected from the group consisting of: Bacillus licheniformis, Bacillus subtilis, Bacillus alkalophilus, Bacillus amyloliquefaciens, Bacillus brevis, Bacillus circulans, Bacillus clausii, Bacillus coagulans, Bacillus firmus, Bacillus jautus, Bacillus lentus, Bacillus megaterium, Bacillus pumilus, Bacillus stearothermophilus, Bacillus thuringiensis, and Bacillus velezensis.

15. The method of claim 1, wherein said expression construct for a gene encoding a protein of interest has been introduced into the Bacillus host cell by genetic modification.

16. The method of claim 1, wherein said at least one feed solution comprises at least one carbon source.

17. A Bacillus host cell culture obtainable by the method of claim 1.
